(19) United States
(12) Patent Application Publication
(10) Pub. No.: US 2010/0010634 A1
(43) Pub. Date: Jan. 14, 2010

(54) Biocompatible intervertebral spacer

(76) Inventor: Radames Binotto, Torreglia (IT)

Correspondence Address: Themis Law, 7660 Fay Ave Ste H-535, La Jolla, CA, 92037, US

(21) Appl. No.: 12/373,889

(22) PCT Filed: Jul. 18, 2006
(86) PCT No.: PCT/IT06/00542

§ 371 Date: Jan. 26, 2009

(51) Int. Cl. A61F 2/44

(52) U.S. Cl. 623/17.16

(57) ABSTRACT

An intervertebral spacer configured for insertion between two vertebrae includes two opposite plates having one or more protruding parts for immobilization in the intervertebral space, a shock absorbing element, and a rigid spacer element positioned and constrained between the plates. The rigid spacer element permits small relative movements between the plates, which are countered by the shock absorbing element.

The present invention concerns medical devices for spinal neurosurgery, and in particular concerns a new intervertebral spacer.

### BACKGROUND

Patients with vertebral pathologies, for example of degenerative or traumatic origin, must often undergo surgeries to restore or correct the connection between the vertebrae of the spinal column.

In this case the use of interosseus spacers, i.e. elements having form and dimensions such as to be inserted in the intervertebral spaces to restore the correct position and/or distance of the vertebrae while guaranteeing appropriate mobility and strength, is known.

In surgical operations for implanting one or more intervertebral spacers, the expression primary immobilization is used, meaning the initial position at the time of the operation, and secondary immobilization, meaning the intersomatic fusion which guarantees that the implant remains correctly positioned also in the long term.

Many of the known spacers have low mechanical strength and a fairly poor level of intersomatic fusion, consequently their positioning in the intervertebral space is not stable.

In such case, the spacer implanted in the vertebral column may be displaced from the required position, with consequent serious problems for the patient.

It is known that, to partly remedy said drawbacks, particularly in the case of implants of intervertebral spacers on two or more levels, i.e. involving three or more vertebrae, additional connection plates between the intervertebral spacers must be used, which are located in an anterior position with respect to the vertebral column and fixed to the vertebrae by means of screws.

The operation undergone by the patient is therefore extremely invasive and traumatic and recovery times are very long.

Spacers are known that are made of material of organic or synthetic origin of high biocompatibility, i.e. having characteristics similar to those of human tissue such as not to prevent intersomatic fusion.

Ceramic intervertebral implants are known, used for example to fill the spaces created in the vertebral column in the case of an operation, in which the vertebral disc is removed, for example, in slipped disc operations.

Interosseus spacers particularly used for cervical decompression, aimed at correcting vertebral pathologies of the cervical area and correctly re-locating the vertebrae without touching the spinal channel and without preventing natural drainage between adjacent vertebrae, are also known.

A further drawback of the known spacers consists in their limited adaptability to the intervertebral spaces of the patient, consequently also limiting mobility of the vertebral column in the vicinity of the implant area.

### SUMMARY

To remedy all the above drawbacks a new type of biocompatible vertebral spacer has been designed and produced.

The object of the new spacer is to guarantee high adaptability in the intervertebral space.

A further object of the present invention is to permit a stable initial positioning, i.e. to allow primary immobilization.

A further object is to permit a high level of spinal intersomatic fusion, i.e. provide secondary immobilization.

A further, important object of the present invention is not to require the use of plates and/or screws for connection and fixing to the vertebrae, thus making the operation less invasive for the patient.

A further object is to obtain a mechanical strength comparable with that of the vertebrae.

The new spacer is particularly suitable for application in the cervical region.

These and other objects, direct and complementary, are achieved by a biocompatible vertebral spacer constructed according to the principles of the present invention, which is suitable for insertion in the intervertebral space, i.e. between two vertebrae, and in which the two opposite sides facing the related vertebrae comprise protruding parts configured for permitting and guaranteeing primary and secondary immobilization of the spacer.

In particular, the new spacer comprises two opposing and preferably specular plates, between which at least one shock absorbing element, made of silicone for example, and at least one rigid spacer element are positioned. These plates, made of a biocompatible material with a high mechanical strength, comparable to that of the vertebrae, comprise, on the outer side facing the vertebrae, protruding parts suitable for permitting primary and secondary immobilization of the spacer.

These protruding parts allow the spacer to be blocked in a stable manner in the required position without the aid of plates and screws, particularly in the case of an implant of intervertebral spacers on two or more levels, i.e. involving three or more vertebrae.

In particular, each of these plates includes at least one protruding part suitable for insertion in cavities provided on the vertebrae, such to guide a correct positioning of the spacer.

More particularly, this protruding part has an outer surface that is preferably convex, for example hemispherical, in order not to obstruct insertion of the spacer in the intervertebral space.

These plates also include, on the outer faces, one or more protrusions or teeth, which prevent the spacer from subsequently coming out of the established position.

These protrusions or teeth are distributed in corresponding or non-corresponding positions on each of the two plates, so that the spacer is, as a whole, symmetrical or non-symmetrical. For insertion of the new spacer, the surgeon firstly mills the two vertebrae, to create the cavities suitable for housing the convex protruding parts of the plates.

The new spacer is then positioned in the intervertebral space, so that these convex protruding parts are inserted in the cavities and provide primary immobilization.

The above described teeth ensure that the spacer does not come out of the required position.

The central shock absorbing element has a substantially parallelepiped shape and is positioned and constrained between the two plates.

The central shock absorbing element may be made of silicone or of similar medical plastic materials, natural or synthetic polymers, including materials suited for grafting a differentiated cellular material suitable for activating bone growth and/or growth of cartilage or other. The central shock element may also be produced using CAT or MIR data with stereolithographic systems, sintering or similar, and may imitate the bone trabeculae and be custom-made.

These plates, therefore, if not subject to any type of stress, are arranged parallel, whereas, when constrained in the intervertebral space, these plates can arrange themselves in a converging manner, thus adapting to the form of the spacer, due to the central shock absorbing element, which can sustain small deformations.

The rigid spacer element is constrained between said two plates and is contained, for example, in the central shock absorbing element.

The rigid spacer element, which, in the preferred embodiment, has a substantially spherical form, is located in a substantially central position between the two plates and substantially has the function of permitting the relative movements between the two plates, while the shock absorbing element counters such movements.

The material used for the construction of all the parts making up the new spacer has mechanical characteristics similar to those of the adjacent vertebrae, such to integrate perfectly with the human tissue and guarantee adequate mechanical strength. In this regard, it is envisaged that the plates will be made of a metal, such as titanium, or a plastic polymeric material, for example caprolactone or its derivatives.

More generally, the materials that can be used for the plates are: hydroxylapatite mixed with plasma and gelatins of various origins and growth factors, ceramic bioglass suitable for bone fusion mixed with gelatins of various origins and growth factors, metal implantology products, for example medical steel, ceramic materials, etc.

It is preferable for the central shock absorbing element to be made of silicone, whereas it is expedient to make the rigid spacer element of titanium or other biocompatible synthetic material with high mechanical strength, such as a synthetic diamond or a synthetic ruby.

It is furthermore envisaged that the plates and the central shock absorbing element will be provided with openings for the passage of cells and to permit growth of the bone trabeculae inside them, thus favoring secondary immobilization of the spacer and fusion with the adjacent bodies.

### BRIEF DESCRIPTION OF THE DRAWINGS

The constructive features of the new spacer will be better clarified by the following description with reference to the drawings, attached as a non-limiting example.

FIGS. 1 and 2 show respectively, in three-dimensional views, the upper (Ps) and lower (Pi) plates of a spacer (O) according to an embodiment of the present invention.

FIG. 3 shows the spherical rigid spacer element (S) suitable for insertion in the housing (A1) provided in the central shock absorbing element (A), which is shown in a three-dimensional view in FIG. 4.

FIG. 5 shows a cross-section along the vertical central section plane (π) of the spacer (O) having the plates of FIGS. 1 and 2, which is shown completely assembled shown in FIG. 6.

FIG. 7 shows a three-dimensional view of an embodiment of a spacer (O) according to the present invention.

FIG. 8 shows an embodiment of spacer (O) according to present invention having rigid spacer element (S) integral with the upper plate (Ps).

### DETAILED DESCRIPTION

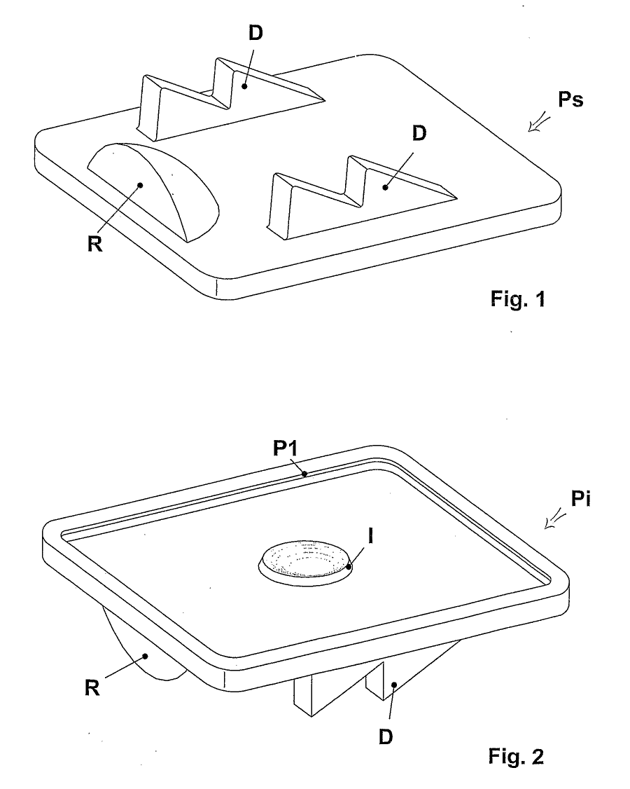

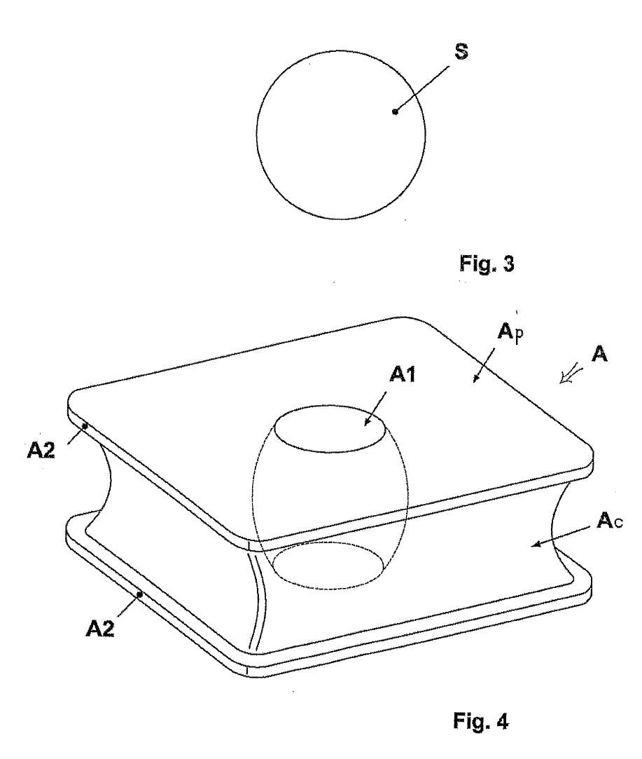

According to the preferred embodiment shown in FIGS. 1-6, the new biocompatible vertebral spacer (O) comprises two opposing and substantially specular plates (Pi, Ps), between which at least one shock absorbing element (A) is positioned, which may be made of silicone, and at least one rigid spacer element (S).

Plates (Pi, Ps), made of biocompatible material with high mechanical strength, comparable to that of the vertebrae, comprise on their outer faces one or more protruding parts (R, D) suitable for permitting primary and secondary immobilization of the spacer.

In particular, each of plates (Pi, Ps) comprises at least one protruding part (R), substantially convex in shape, and suitable for insertion in cavities provided on the vertebrae for correct positioning of the spacer (O).

Plates (Pi, Ps) also comprise, on their outer faces, one or more teeth (D) suitable for preventing the spacer (O) from subsequently coming out of the established position.

A central shock absorbing element (A), positioned between plates (Pi, Ps), has a substantially parallelepiped shape, with opposing faces (Ap) substantially flat and a lateral surface (Ac) curving inwards. In particular, it is envisaged that lateral wall (Ac) may be concave, arcuate or include two or more converging faces.

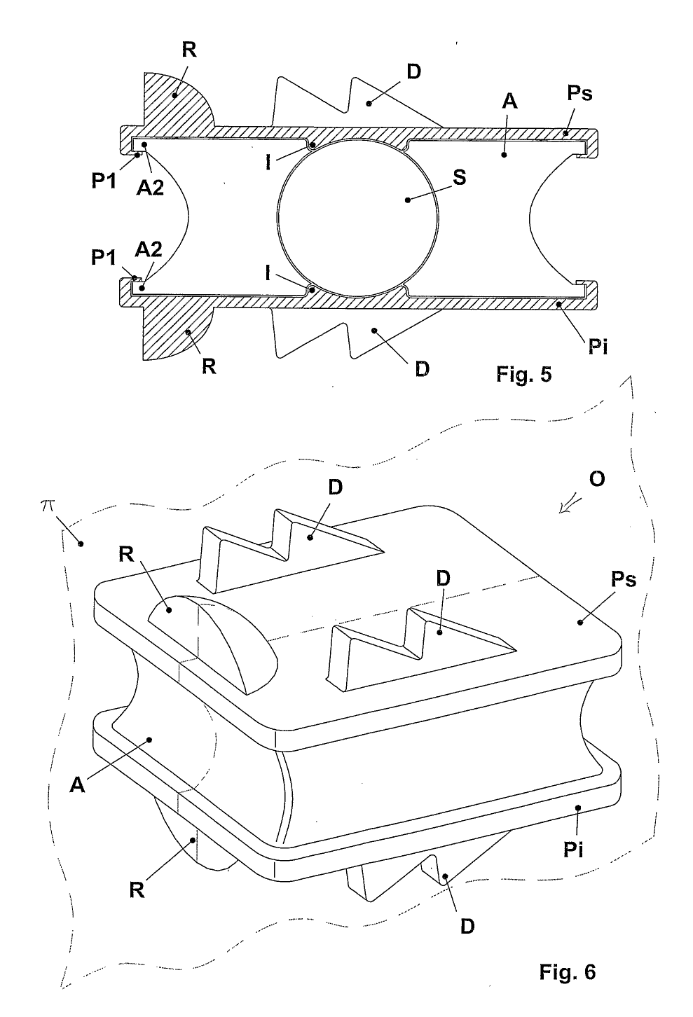

Central shock absorbing element (A) is connected, at the top and bottom, to plates (Pi, Ps), as can be seen in FIG. 5. Said shock absorbing element (A) comprises, at the top and bottom, a protruding perimeter edge (A2) suitable for insertion below the folded-in edge (P1) of plates (Pi, Ps).

Rigid spacer element (S) may have a spherical shape and is inserted in a housing (A1) obtained inside said central shock absorbing element (A).

Said rigid spacer element (S) is furthermore constrained between plates (Pi, Ps), being housed in concave dome-shaped seats (I), positioned on the surface facing the inside of said plates (Pi, Ps), in a substantially central position and corresponding to said housing (A1) of said shock absorbing element (A).

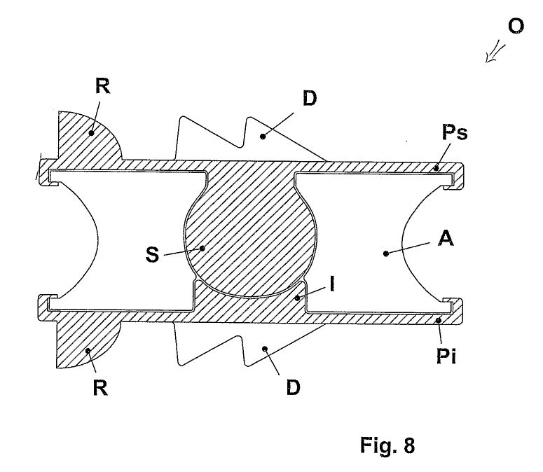

In the embodiment shown in FIG. 8, spacer (O) comprises two opposing and preferably specular plates (Pi, Ps) and rigid spacer (S) is integral with one of the two plates (Pi, Ps), for example with the upper plate (Ps), and is housed in a seat (I) integral with the opposite plate (Pi).

In this configuration, plates (Pi, PS) are pivoting with respect to each other, i.e. they can be arranged in a reciprocal non-parallel position.

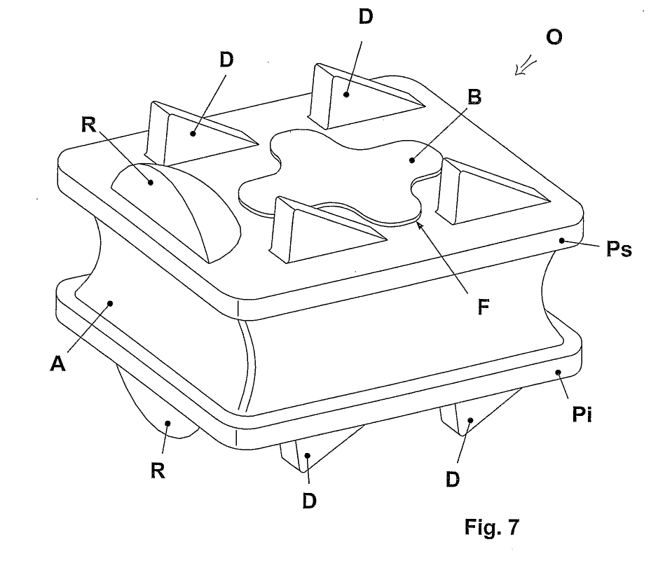

In the embodiment shown in FIG. 7, each of said plates (Pi, Ps) comprises a substantially central seat or hole (F) for the insertion of protruding parts (B) of said central shock absorbing body (A), suitable for guaranteeing assembly of the spacer (O).

Therefore, with reference to the preceding description and the illustrations, the following claims are made.

What is claimed is:

1.-12. (canceled)

13. An intervertebral spacer comprising:
a plurality of opposing plates;
one or more protrusions extending from faces of the opposing plates in contact with vertebrae;
a shock absorbing element disposed between the opposing plates; and
a rigid spacer element disposed between the opposing plates and contained by the shock absorbing element,
wherein the shock absorbing element and the rigid spacer element are disposed such to provide a support between the vertebrae while allowing relative movements of the opposing plates,
wherein the rigid spacer element is shaped to allow converging movements of the opposing plates, and
wherein the shock absorbing element is shaped to counter the converging movements of the opposing plates.

14. The intervertebral space of claim 13, wherein there are two opposing plates disposed parallel and symmetrically with respect to the shock absorbing element.

15. The intervertebral spacer of claim 13, wherein one or both of the opposing plates have a perimeter edge that is partially or totally folded inwards, and wherein the shock absorbing element has one or more perimeter edges that are inserted within the folded perimeter edge of the one or more opposing plates, thereby coupling the perimeter edge of the more or more opposing plates with the one or more perimeter edges of the shock absorbing element.

16. The intervertebral spacer of claim 13, wherein the shock absorbing element has a substantially parallelepiped shape with inwardly curving side walls, and wherein the plurality of opposing plates are disposed against upper and lower faces of the shock absorbing element.

17. The intervertebral spacer of claim 13, wherein the rigid spacer element is inserted in a housing disposed within the shock absorbing element in a substantially central position.

18. The intervertebral spacer of claim 13, wherein the rigid spacer element is housed in a seat positioned on an inner face of one or more of the opposing plates.

19. The intervertebral spacer of claim 13, wherein the rigid spacer element has a substantially spherical shape.

20. The intervertebral spacer of claim 13, wherein the rigid spacer element is integral with one of the opposing plates and is at least partially received in a seat positioned on an inner face of another one of the opposite plates.

21. The intervertebral spacer of claim 13, wherein one or more of the opposing plates comprise one or more seats or openings for inserting one or more protruding portions of the shock absorbing element, thereby providing a connection between the shock absorbing element and one or both of the opposing plates.

22. The intervertebral spacer of claim 13, wherein one or more of the opposing plates and of the shock absorbing element comprises one or more openings or ducts for a passage of living cells.

23. The intervertebral spacer of claim 13, wherein at least one of the one or more protrusions is at least partially convex in shape.

24. The intervertebral spacer of claim 13, wherein at least one of the one or more of protrusions is a substantially triangular tooth.

25. The intervertebral spacer of claim 13, wherein the shock absorbing element comprises a resilient gel or gelatinous material.

26. The intervertebral spacer of claim 25, wherein the gel or gelatinous material is a silicone material.

27. The intervertebral spacer of claim 13, wherein the intervertebral spacer comprises one or more materials selected from the group consisting of hydroxylapatite mixed with plasma and gelatins of various origins and growth factors, ceramic bioglass suitable for bone fusion mixed with gelatins of various origins and growth factors, metal implantology products, titanium, steel, ceramic materials, elastic medical plastic silicone materials, synthetic and natural polymers, tissues, resins, polyethylene, and other materials suitable for stimulating bone or cartilage fusion.
